(19) United States
(12) Patent Application Publication
(10) Pub. No.: US 2013/0196374 A1
(43) Pub. Date: Aug. 1, 2013

(54) CIS-ACTING ELEMENT AND USE THEREOF

(71) Applicant: Hiroaki Koishihara, Nagoya-shi (JP)

(72) Inventor: Hiroaki Koishihara, Nagoya-shi (JP)

(73) Assignee: TOYOTA JIDOSHA KABUSHIKI KAISHA, Toyota-shi, Aichi (JP)

(21) Appl. No.: 13/877,083

(22) PCT Filed: Sep. 29, 2011
(86) PCT No.: PCT/JP2011/072450

§ 371 Date: Mar. 29, 2013

(30) Foreign Application Priority Data
Sep. 30, 2010 (JP) 2010-222131

(51) Int. Cl. C12P 21/00

(52) U.S. Cl. CPC C12P 21/00
USPC 435/69.1

(57) ABSTRACT

The expression level of a desired gene is significantly improved when a filamentous fungus or the like is used as a host cell. The cis-acting element according to the present invention has a region in which the XlnR/Ace2-binding sequence (ggctaa) and the Hap complex-binding sequence (ccaat) are arranged with a spacer sequence of 0 to 100 nucleotides between them. Also, a transformant having the cis-acting element according to the present invention is cultured in a xylan-containing medium, for example, so that the expression level of a desired gene can be significantly improved.

### TECHNICAL FIELD

The present invention relates to: a cis-acting element that positively regulates the expression of a desired gene upon protein production using a filamentous fungus as a host; a nucleic acid construct, an expression vector, and a transformed cell having the cis-acting element; and a method for producing a substance using the cis-acting element.

### BACKGROUND ART

Filamentous fungi belonging to the genus Aspergillus, the genus Trichoderma, or the like are known as microorganisms used for producing various fermented foods, for a substance production (in the fermentation industry), such as for pharmaceutical products, and the like. Among filamentous fungi, fungi of the genus Penicillium and fungi of the genus Cephalosporium are known to produce antibiotics. Moreover, among filamentous fungi, fungi of the genus Trichoderma are known to produce cellulase, and fungi of the genus Aspergillus are known to produce protease and lactase.

Among substances to be produced by filamentous fungi, enzymes such as cellulase and protease are gene products, so that productivity can be directly improved by improving the expression levels of the genes. In other words, to improve the productivity of a protein such as an enzyme described above, development of a means to improve the expression level of a predetermined gene within a filamentous fungus is desired.

Non-patent Document 1 discloses a method for improving the expression of a foreign gene in Trichoderma reesei. The method disclosed in Non-patent Document 1 involves preparing a modified promoter by modifying a cellobiohydrolase gene (cbh1) promoter that it lacks a region containing a glucose repressor binding site and contains a repeatedly-ligated 200-bp region containing a CCAAT box and an Ace2 binding site.

In Non-patent Document 1, transformed Trichoderma reesei was prepared by ligating a reporter gene to a site downstream of the modified promoter, the thus obtained transformant was cultured in a lactose-containing medium, and then the activity of the modified promoter was evaluated by reporter assay. As a result, the promoter containing the above 200-bp region repeatedly ligated 4 times had improved promoter activity to a level that was about 1.4 times greater than that of a promoter having only one such region. In addition, a promoter containing the 200-bp region repeatedly ligated 6 times had activity almost equivalent to that of a promoter containing the 200-bp region repeatedly ligated 4 times.

As described above, Non-patent Document 1 provides a means for increasing the amount of a foreign gene in Trichoderma reesei to a maximum level that was about 1.4 times greater than the conventional level. However, a substance production using filamentous fungi is required to yield even better productivity, and thus the expression level of the gene of interest is required to be further significantly improved. Furthermore, a substance production using filamentous fungi is required to improve the expression level of the gene as described above while keeping production costs at low levels.
Non-patent Document 1: Acta. Biochim. Biophys. Sin. (2008): 158-165

### DISCLOSURE OF THE INVENTION

### Problems to Be Solved by the Invention

Therefore, an object of the present invention is to provide: a novel cis-acting element capable of significantly improving the expression level of a desired gene when a filamentous fungus or the like is used as a host cell; a nucleic acid construct, an expression vector, and a transformed cell having the cis-acting element; and a method for producing a substance using the cis-acting element.

### Means for Solving the Problem

As a result of intensive studies to achieve the above object, the present inventors have discovered that a relatively short region (compared with the above 200-bp region disclosed in Non-patent Document 1) having the XlnR/Ace2-binding sequence (ggctaa) and the Hap complex-binding sequence (ccaat) serves as a cis-acting element to enable high-level expression of a gene downstream thereof, and in particular, the region enables the expression of a gene downstream thereof at an even higher level in the presence of xylan. Thus, the present inventors have completed the present invention.

The present invention encompasses the following (1) to (11).

(1) A cis-acting element, comprising a region in which the XlnR/Ace2-binding sequence (ggctaa) and the Hap complex-binding sequence (ccaat) are arranged with a spacer sequence of 0 to 100 nucleotides between them.
(2) The cis-acting element according to (1), consisting of the nucleotide sequence of nnnggctaannnnnnccaatnnnnnn (where n denotes an arbitrary nucleotide selected from adenine, cytosine, guanine, and thymine: SEQ ID NO: 3).
(3) The cis-acting element according to (1), comprising a plurality of the regions repeated via linker sequences.
(4) The cis-acting element according to (3), wherein the number of repetitions of the above region ranges from 1 to 50.
(5) A nucleic acid construct, comprising the cis-acting element of any one of (1) to (4) and a promoter region.
(6) An expression vector, comprising the cis-acting element of any one of (1) to (4) and a promoter region located downstream of the cis-acting element.
(7) The expression vector according to (6), further comprising a gene located downstream of the above promoter region.
(8) A transformant, wherein the cis-acting element of any one of (1) to (4) is incorporated into a site upstream of a promoter region in a desired gene.
(9) The transformant according to (8), wherein the desired gene is a foreign gene.
(10) The transformant according to (8), wherein a filamentous fungus is used as a host cell.
(11) A method for producing a substance, comprising culturing the transformant of any one of (8) to (10), and recovering a target substance from a medium and/or the transformant after culture.
(12) The method for producing a substance according to (11), comprising culturing the above transformant in a xylan-containing medium.
(13) The method for producing a substance according to (11), comprising culturing the above transformant in a wheat bran medium.
(14) The method for producing a substance according to (11), wherein the above target substance is a protein that is encoded by a gene in which the expression of the gene is enhanced by the above cis-acting element.

This description includes part or all of the contents as disclosed in the description and/or drawings of Japanese Patent Application No. 2010-222131, which is a priority document of the present application.

### Effects of the Invention

With the cis-acting element according to the present invention, the expression level of a gene located downstream thereof can be significantly improved. The cis-acting element according to the present invention is incorporated into an expression control region in an endogenous gene of a filamentous fungus, so that the endogenous gene can be expressed at a high level. Furthermore, with the use of an expression vector having the cis-acting element according to the present invention, a gene incorporated into the expression vector can be expressed at a high level within a filamentous fungus.

With the method for producing a substance according to the present invention, the expression level of a predetermined gene is improved by the use of the above cis-acting element, and thus excellent productivity can be achieved. Specifically, the method for producing a substance according to the present invention can significantly improve the productivity of: a protein to be encoded by a gene the expression of which is accelerated by the above cis-acting element; and/or various substances in which the protein is involved.

### BRIEF DESCRIPTION OF THE DRAWINGS

FIG. 1 is a flow chart showing the process for preparing entry clones pENTR-Ptefl, pENTR-Pteflil2, and pENTR-Ptefli6.

FIG. 2 is a flow chart showing the process for preparing entry clones pENTR-PamyB and pENTR-GUS.

FIG. 3 is a flow chart showing the process for constructing a pDEST-Ptefl gene expression vector.

FIG. 4 is a flow chart showing the process for constructing a pDEST-Ptefi6 gene expression vector.

FIG. 5 is a flow chart showing the process for constructing a pDEST-Ptefil2 gene expression vector.

FIG. 6 is a flow chart showing the process for constructing a pDEST-PamyB gene expression vector.

FIG. 7 is a characteristic diagram showing the results of determining GUS activity when transformants Ptefl, Ptefil2, Ptefi6, and PamyB were cultured by solid culture, bran liquid culture, and DPY liquid culture.

### BEST MODES FOR CARRYING OUT THE INVENTION

Hereafter, the present invention is described in detail.

The cis-acting element according to the present invention comprises a region having a predetermined nucleotide sequence and has a function to accelerate transcription from a promoter region in a gene located downstream thereof. Specifically, the cis-acting element according to the present invention comprises a region wherein the XlnR/Ace2-binding sequence (ggctaa) and the Hap complex-binding sequence (ccaat) are arranged with a nucleic acid (spacer region) of 0 to 100 nucleotides between them. In other words, the cis-acting element according to the present invention comprises the region denoted as 5′-ggctaaNₘccaat-3′ (SEQ ID NO: 1) or 5′-ccaatNₘggctaa-3′ (SEQ ID NO: 2). Specifically, the cis-acting element according to the present invention may have, from the 5′ side, the XlnR/Ace2-binding sequence (ggctaa) and the Hap complex-binding sequence (ccaat) in such order, or from the 5′ side, the Hap complex-binding sequence (ccaat) and the XlnR/Ace2-binding sequence (ggctaa) in such order.

Here, “N” is an arbitrary nucleotide selected from adenine, cytosine, guanine, and thymine, and “m” is an integer between 0 and 100. Specifically, in the above cis-acting element, Nₘ is 0 to 100 nucleotides in length and is composed of an arbitrary nucleotide sequence. In particular, the length of Nₘ is not particularly limited, but can range from 1 to 100 nucleotides, preferably ranges from 1 to 50 nucleotides, more preferably ranges from 1 to 20 nucleotides, and most preferably ranges from 3 to 10 nucleotides, for example. A case in which “m” is 0 refers to a case in which the XlnR/Ace2-binding sequence (ggctaa) and the Hap complex-binding sequence (ccaat) are directly linked without any spacer region. Specifically, the cis-acting element according to the present invention can be a region in which the XlnR/Ace2-binding sequence (ggctaa) and the Hap complex-binding sequence (ccaat) are directly linked. Alternatively, the cis-acting element according to the present invention can comprise a region in which the XlnR/Ace2-binding sequence (ggctaa) and the Hap complex-binding sequence (ccaat) are arranged with a nucleic acid (spacer region) of 1 to 100 nucleotides between them. With the length of a region consisting of Nₘ sets within the above range, excellent transcriptional activity can be achieved.

The cis-acting element according to the present invention preferably has a structure in which the above region containing the XlnR/Ace2-binding sequence (ggctaa), the Hap complex-binding sequence (ccaat), and the spacer region is repeated multiple times. Here, the phrase “repeated multiple times” refers to a situation in which the above sequences (composing the region) are arranged in tandem with linker sequences each having a predetermined nucleotide length. The term “linker sequence” refers to a region with a predetermined nucleotide length, which is located between adjacent pairs of regions. The nucleotide length of such a linker sequence is not particularly limited and may range from 1 to 100 nucleotides in length in a manner similar to that of Nₘ above.

An example of the cis-acting element according to the present invention is an element comprising a region that consists of nnnggctaannnnnnccaatnnnnnn (5′ side→3′ side: SEQ ID NO: 3) (where n is an arbitrary nucleotide selected from adenine, cytosine, guanine, and thymine). Six (6) nucleotides located between “ggctaa” and “ccaat” in the region shown in SEQ ID NO: 3 form a spacer region. Three nucleotides on the 3′ side and six nucleotides on the 5′ side in the region shown in SEQ ID NO: 3 are linker sequences. More specifically, an example of the cis-acting element according to the present invention is the region consisting of ttaggctaaacgtacccaatgataag (SEQ ID NO: 4). In addition, in the region shown in SEQ ID NO: 4, six nucleotides located between “ggctaa” and “ccaat” form a spacer region, and three nucleotides on the 3′ side and six nucleotides on the 5′ side are linker sequences.

Moreover, in the cis-acting element according to the present invention, when the above region comprising the XlnR/Ace2-binding sequence (ggctaa), the Hap complex-binding sequence (ccaat), and a spacer region is repeated multiple times, the number of the region is not limited, and can range from 1 to 50, preferably range from 2 to 30, and more preferably range from 6 to 24. When the number of the above region is lower than the above range, the effect of improving transcriptional activity may not be sufficiently exhibited. Also, the higher the number of repetitions of the above region, the more improved the transcriptional activity. When the number of repetitions of the region is higher than the above range, transcriptional activity may not be further improved.

As described above, 1 or a plurality of the above regions are located so that the cis-acting element according to the present invention can improve transcriptional activity from the promoter located downstream. Here, the term “downstream” refers to the transcriptional direction; that is, the direction of a sense strand from the 5′ side to the 3′ side.

Through the use of the cis-acting element according to the present invention, a nucleic acid construct having an expression control region excellent in transcriptional activity can be provided. In addition, the effect of improving transcriptional activity exhibited by the cis-acting element can be evaluated by ligating a reporter gene to the above nucleic acid construct and then detecting the expression of the reporter gene. Examples of such a reporter gene that can be used herein include, but are not limited to, a luciferase (LUC) gene and a β-glucuronidase (GUS) gene. Assay using these reporter genes can also be performed by appropriately modifying conventionally known protocols.

Here, the term “nucleic acid construct” refers to a nucleic acid comprising the cis-acting element having 1 or a plurality of the above regions, and a promoter region located downstream of the cis-acting element. The nucleic acid construct can also be constructed so that it has restriction enzyme recognition sequences on both ends, for example. The nucleic acid construct can also be incorporated into a conventionally known expression vector, for example. Specifically, through incorporation of the above cis-acting element according to the present invention into an expression vector that enables the expression of a desired gene, an expression vector capable of improving gene expression at the transcriptional level can be provided.

The expression vector can be constructed by incorporating the above cis-acting element into all conventionally known expression vectors that are mainly used for transformation of host cells. Furthermore, the expression vector having the above cis-acting element may be in a form such that it is introduced into the chromosome of a host cell or in a form such that it is retained outside the chromosome. Also, the expression vector may be any of a plasmid vector, a cosmid vector, a phage vector, and the like. In addition, the expression vector may comprise, in addition to the above cis-acting element and promoter, an enhancer, a selection marker, a replication origin, multiple cloning sites, and the like.

Moreover, when the expression vector is used for transformation of filamentous fungi, examples of a promoter that can be preferably used herein include, but are not particularly limited to, a tef1 promoter (derived from A. oryzae), a cbh1 promoter (derived from T. reesei), and an amyB promoter (derived from A. oryzae), as long as it enables gene expression within host filamentous fungi. Furthermore, as a promoter, in addition to these examples, an ADH3 promoter, a tpiA promoter, an alcA promoter, a taaG2 promoter, a gpdA promoter, or the like can be used.

Through incorporation of a desired gene into the above expression vector having the cis-acting element, a recombinant vector can be constructed. Host cells are transformed with the recombinant vector, so that the gene is transcribed at a high level in the host cells. Host cells to be used herein are not particularly limited and are preferably fungi such as filamentous fungi and are particularly preferably filamentous fungi.

Examples of filamentous fungi that can be used as hosts include, but are not particularly limited to, filamentous fungi of the genus Aspergillus such as Aspergillus nidulans, Aspergillus niger, Aspergillus oryzae, Aspergillus sojae, and Aspergillus glaucus, filamentous fungi of the genus Trichoderma such as Trichoderma reesei and Trichoderma viride, filamentous fungi of the genus Rhizomucor such as Rhizomucor pusillus and Rhizomucor miehei, filamentous fungi of the genus Penicillium such as Penicillium notatum and Penicillium chrysogenum, filamentous fungi of the genus Rhizopus such as Rhizopus oryzae, Acremonium cellulolyticus, Humicola grisea, and Thermoaseus aurantiacus. Particularly preferred hosts include filamentous fungi of the genus Aspergillus and particularly Aspergillus oryzae, and filamentous fungi of the genus Trichoderma and particularly Trichoderma reesei.

As methods for introducing a recombinant vector into a host, various conventionally known methods such as a transformation method, a transfection method, a conjugation method, a protoplast method, an electroporation method, a lipofection method, a lithium acetate method, and the like can be employed.

Examples of a gene to be introduced into a host using a recombinant vector include, but are not particularly limited to, genes encoding various proteins. Examples thereof include an alkali protease gene, an α-amylase gene, an ascorbic acid oxidase gene, an aspartic protease gene, a cellobiohydrolase gene, a cellulase gene, a cutinase gene, an endoglucanase gene, glucoamylase, a β-glucosidase gene, a glyoxal oxidase gene, a laccase gene, a lignin oxidase gene, a lignin peroxidase gene, a lipase gene, a manganese peroxidase gene, a 1,2-α-mannosidase gene, a nuclease gene, a pectin lyase gene, a pectin methylesterase gene, an acid phosphatase gene, a polygalacturonase gene, a xylanase gene, and a β-xylosidase gene. Of these genes, a cellobiohydrolase gene, an endoglucanase gene, and a β-glucosidase gene from fungi of the genus Trichoderma and an amylase gene, a protease gene and a glucoamylase gene from fungi of the genus Aspergillus are preferably used herein.

Meanwhile, the form of the cis-acting element according to the present invention to be introduced together with a desired gene into host cells is not limited as described above. For example, a form to be incorporated into an expression control region of an endogenous gene of a host cell is also applicable herein. A recombinant prepared by incorporating the cis-acting element according to the present invention into an expression control region of an endogenous gene of a host cell is also referred as a transformant as in the case of the above form to be introduced into a host cell together with a desired gene.

For example, the above cis-acting element is inserted upstream of a promoter of an endogenous gene, so that transcriptional activity from the promoter can be improved. Alternatively, for example, a nucleic acid construct comprising the above cis-acting element and promoter is inserted upstream of a coding region of an endogenous gene, so that transcriptional activity of the endogenous gene can be improved.

For insertion of the above cis-acting element or nucleic acid construct into a desired position on the chromosome of a host cell, a conventionally known technique can be employed, such as a technique using a Ku gene-disrupted strain. For example, through homologous recombination using nucleotide sequence information concerning positions to be subjected to insertion, the above one or multiple cis-acting elements or nucleic acid constructs can be inserted. Here, the term “ku gene” refers to a gene encoding a protein required for non-homologous recombination and examples thereof include a ku70 gene and a ku80 gene. Regarding homologous recombination using Aspergillus oryzae ku70 gene-disrupted strain, Aichi Center for Industry and Science Technology, Research Report (7), 90-93, 2008-12 can be referred.

A transformant having the cis-acting element according to the present invention is preferably cultured in a medium containing particularly xylan. When the transformant is cultured in a xylan-containing medium, transcription-accelerating activity is more effectively exhibited by the cis-acting element. This is because an XlnR gene existing in the transformant is expressed at a high level due to xylan contained in the medium, and the XlnR transcription factor sufficiently acts on the XlnR/Ace2-binding sequence (ggctaa) contained in the above cis-acting element.

Here, the term “xylan-containing medium” refers to a medium containing xylan at the detection limit or higher. The concentration of xylan contained in a liquid medium is not particularly limited and can range from 0.1% w/v to 15% w/v, preferably ranges from 0.5% w/v to 12% w/v, and more preferably ranges from 1% w/v to 10% w/v, for example. However, examples of the range of the concentration are not limited thereto. When the concentration of xylan is lower than the above range, the expression of the XlnR gene is not sufficiently induced in a transformant, and thus transcription-accelerating activity cannot be sufficiently achieved by the above cis-acting element. When the concentration of xylan is higher than the above range, a substrate within the liquid medium can absorb fluids to result in a problem such as incomplete culture due to insufficient agitation.

Examples of a xylan-containing medium include particularly a medium containing an herbaceous species such as wheat, rice, or bagasse as a raw material, a medium containing woody species as a raw material, and a medium containing an agricultural product residue or waste as a raw material. A typical example of such a medium containing an herbaceous species as a raw material is a wheat bran medium. For example, when the above listed media containing herbs as raw materials, such as a wheat bran medium, are used, a target substance can be produced at very low cost since expensive components are not contained as raw materials. Here, the term “a target substance (to be produced)” refers to either a protein encoded by a gene to be transcribed at a high level due to the above cis-acting element or a substance in which the protein is involved. The term “substance in which the protein is involved” refers to a metabolite, for example, when the protein is involved as an enzyme in the metabolic pathway. When the protein is cellulase, an example of such a substance, in which the protein is involved, is a sugar resulting from a saccharification reaction conducted using cellulose contained in the medium as a substrate.

### EXAMPLES

Hereafter, the present invention is described in greater detail with reference to the examples, although the technical scope of the present invention is not limited to the examples.

### Example 1

In this Example, the functions of the cis-acting element that the present inventor had independently designed were confirmed based on the expression of a reporter gene.

### Experimental Procedure

### (1) Provision of Restriction Enzyme Sites to Promoter Regions

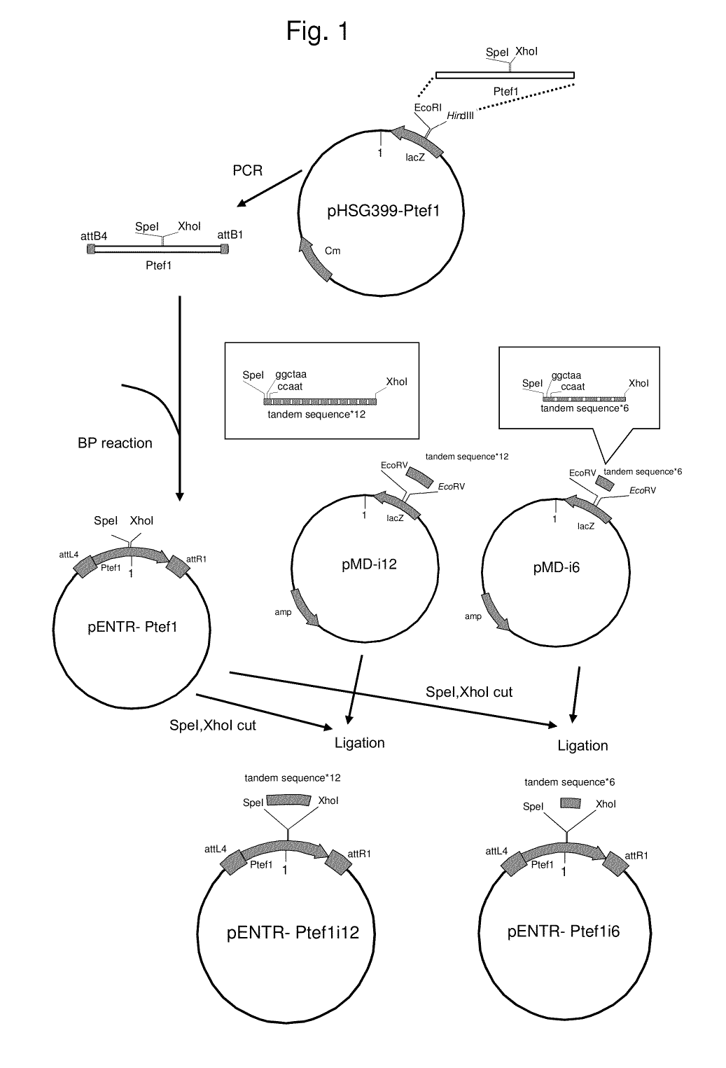

As shown in FIG. 1, a nucleic acid fragment consisting of a nucleotide sequence was synthesized by providing a [Spe I-Xho I] restriction enzyme site to a part in the middle of a 471-bp region upstream of the translation initiation site of translation elongation factor 1 alpha in Aspergillus oryzae. Next, the thus synthesized nucleic acid fragment was introduced into a Hind III-EcoR I site of pHSG399 (Takara Bio Inc) (pHSG399-Ptefl). Next, for the purpose of plasmid construction using a MultiSite Gateway (Invitrogen), gene amplification was performed using pHSG399-Ptefl as a template and a primer pair (A1 and A2) so that the attB4 sequence was provided at the 5′ side end and the attB1 sequence was provided at the 3′ side end of the 471-bp region. The thus amplified fragment was subjected to BP reaction with pDONRP4-P1R, so that an entry clone was prepared (pENTR-Ptefl).

(SEQ ID NO: 5)
A1: 5′-ggggacaactttgtatagaaaagttgtttctagatagcgagag
taaaa-3′
(SEQ ID NO: 6)
A2: 5′-ggggactgcttttttgtacaaacttggtttgaaggtggtgcga
actttg-3′

(2) Synthesis of Fragments with Repeated Enhancer Regions

Next, as shown in FIG. 1, the cis-acting element (ttaggctaaacgtacccaatgataag: (SEQ ID NO: 4); 26 bp) (1 set) containing an enhancer region containing “ggctaa” and a “ccaat” gene expression regulatory region was repeated 12 times (12 sets). A nucleic acid fragment was synthesized so that it consisted of a nucleotide sequence in which a Spe I restriction enzyme site was provided at the 5′ side and an Xho I restriction enzyme site was provided at the 3′ side of the tandem sequence. Furthermore, similarly, a nucleic acid fragment was synthesized to contain a tandem sequence containing the cis-acting element repeated 6 times (6 sets). These nucleic acid fragments were introduced into the EcoR V sites of pMD-simple vectors. (The vector having 12 sets of the cis-acting element is designated as pMD-i12 and the vector having 6 sets of the cis-acting element is designated as pMD-i6.)

### (3) Construction of Various Gene Transfer Vectors Containing Modified Promoters

Next, as shown in FIG. 1, a 320-bp fragment excised with Spe I and Xho I from pMD-i12 was introduced into the Spe I, Xho I site of pENTR-tef1 (pENTR-Pteflil2). Similarly, a 320-bp fragment excised with Spe I and Xho I from pMD-i6 was introduced into the Spe I, Xho I site of pENTR-tef1 (pENTR-Ptefli6).

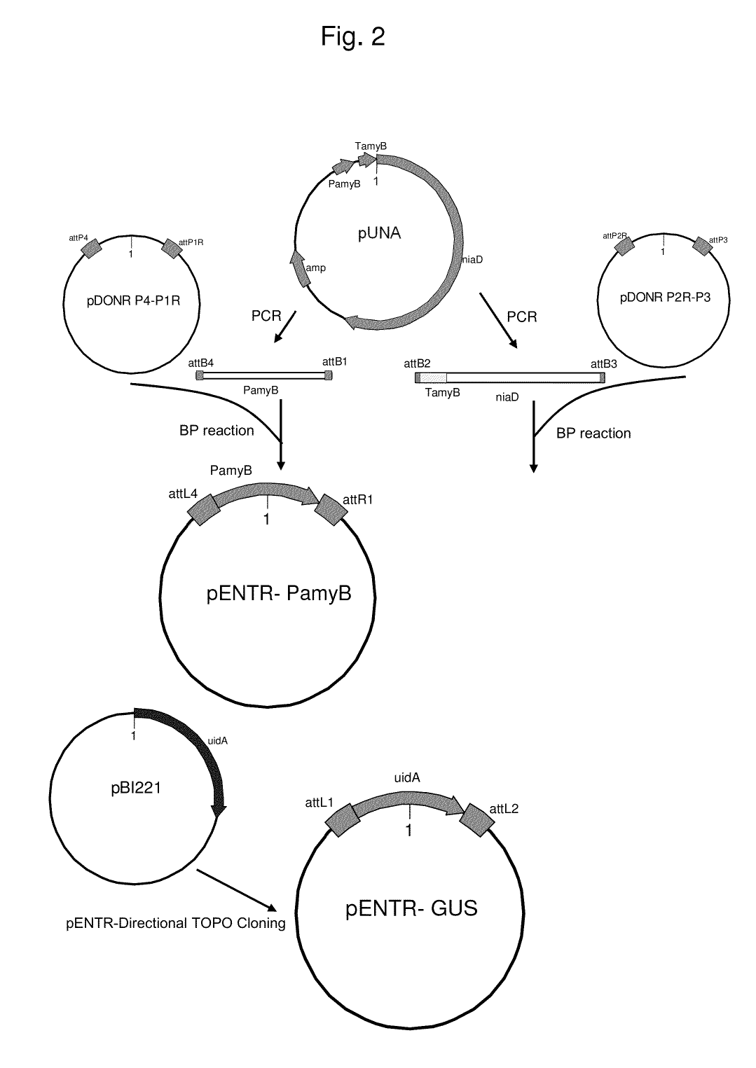

Next, as shown in FIG. 2, a nucleic acid fragment having the attB4 sequence at the 5′ side end and the attB1 sequence at the 3′ side end of an amyB promoter site was amplified by PCR using as a template plasmid pUNA (source: Kitamoto laboratory, the University of Tokyo) containing a promoter and a terminator of an Aspergillus oryzae-derived amyB gene and a nitrate reductase gene (niaD) and a primer pair (B1 and B2). The thus obtained nucleic acid fragment was subjected to BP reaction with pDONRP4-P1R, so that an entry clone was prepared (pENTR-PamyB).

Also, as shown in FIG. 2, a nucleic acid fragment containing a translation region of a βglucuronidase gene was amplified by PCR using as a template a plasmid pBI221 (Clontech) containing β glucuronidase (uidA) and a primer pair (C1 and C2). An entry clone was prepared (pENTR-GUS) using the thus obtained nucleic acid fragment and pENTR Directional TOPO Cloning Kits (Invitrogen).

Furthermore, as shown in FIG. 2, a nucleic acid fragment having the attB2 sequence at the 5′ side end and the attB3 sequence at the 3′ side end of a region containing the amyB terminator and the nitrate reductase gene (niaD) was amplified by PCR using the pUNA as a template and a primer pair (D1 and D2). The thus obtained nucleic acid fragment was subjected to BP reaction with pDONRP2R-P3, so that an entry clone containing the amyB terminator and the nitrate reductase gene (niaD) was prepared (pENTR-niaD).

(SEQ ID NO: 7)
B1: 5′-ggggacaactttgtatagaaaagttgttccagtgaattcatgg
tgttttg-3′
(SEQ ID NO: 8)
B2: 5′-ggggactgcttttttgtacaaacttggaaatgccttctgtggg
gtttatt-3′
(SEQ ID NO: 9)
C1: 5′-atgttacgtcctgtagaaacc-3′
(SEQ ID NO: 10)
C2: 5′-tcattgtttgcctccctgctg-3′
(SEQ ID NO: 11)
D1: 5′-ggggacagctttcttgtacaaagtgggtgatctgtagtagctc
gtgaag-3′
(SEQ ID NO: 12)
D2: 5′-ggggacaactttgtataataaagttggaagctttggatttcct
acgtct-3′

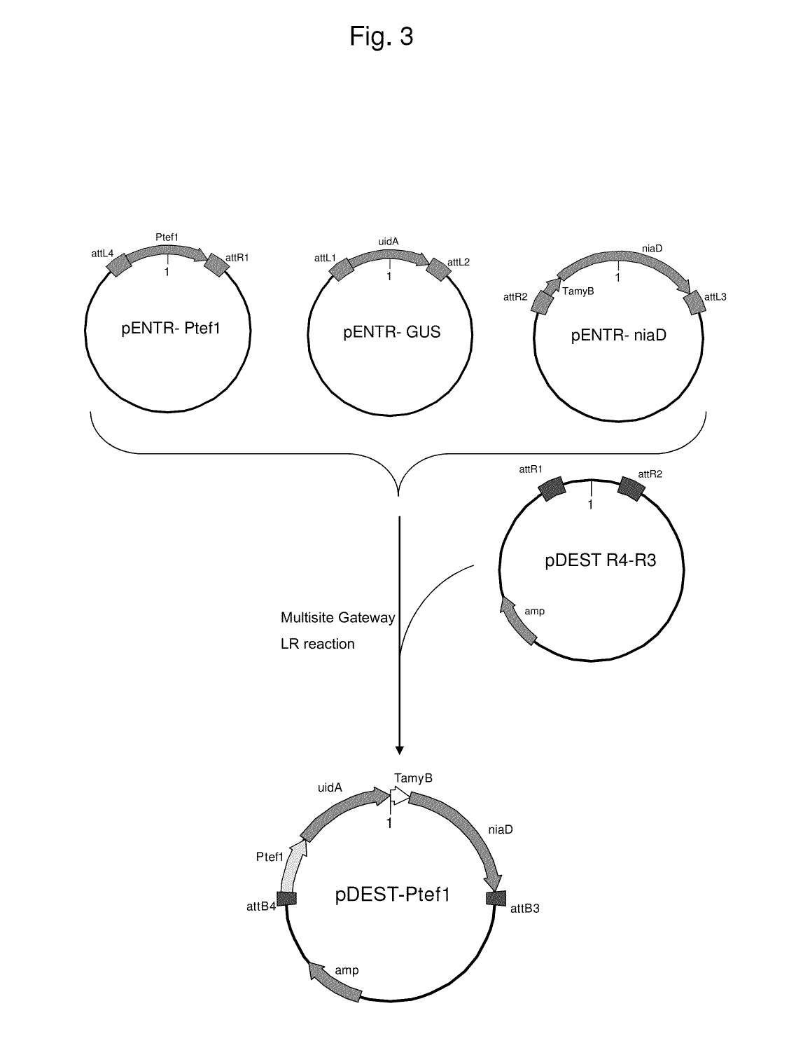

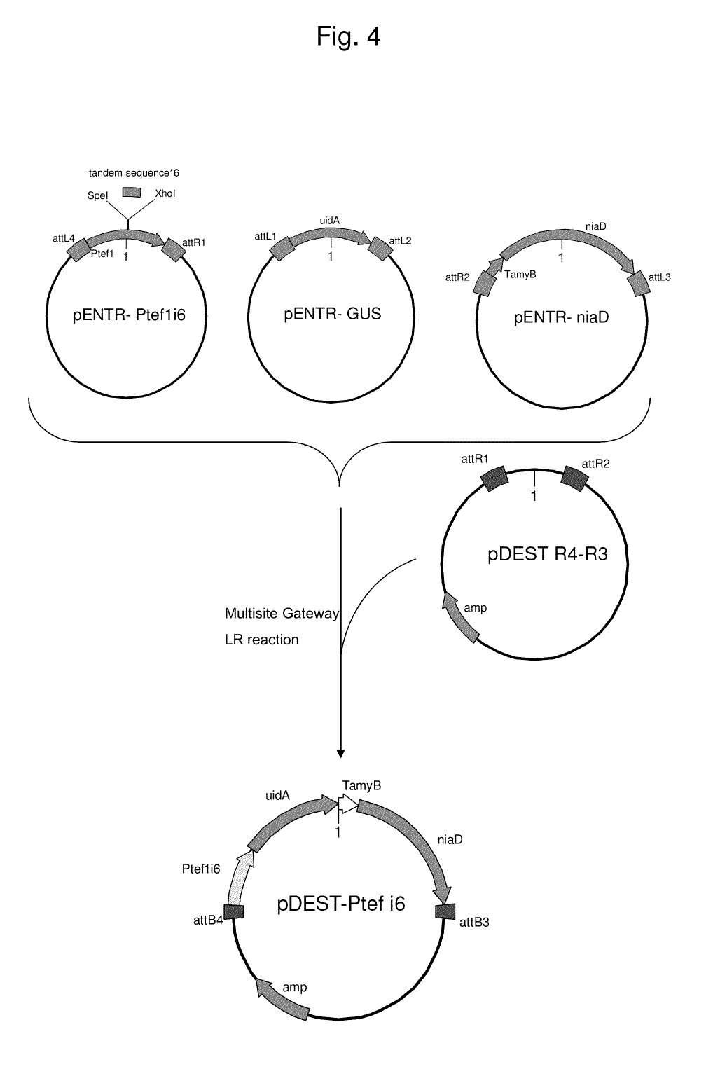

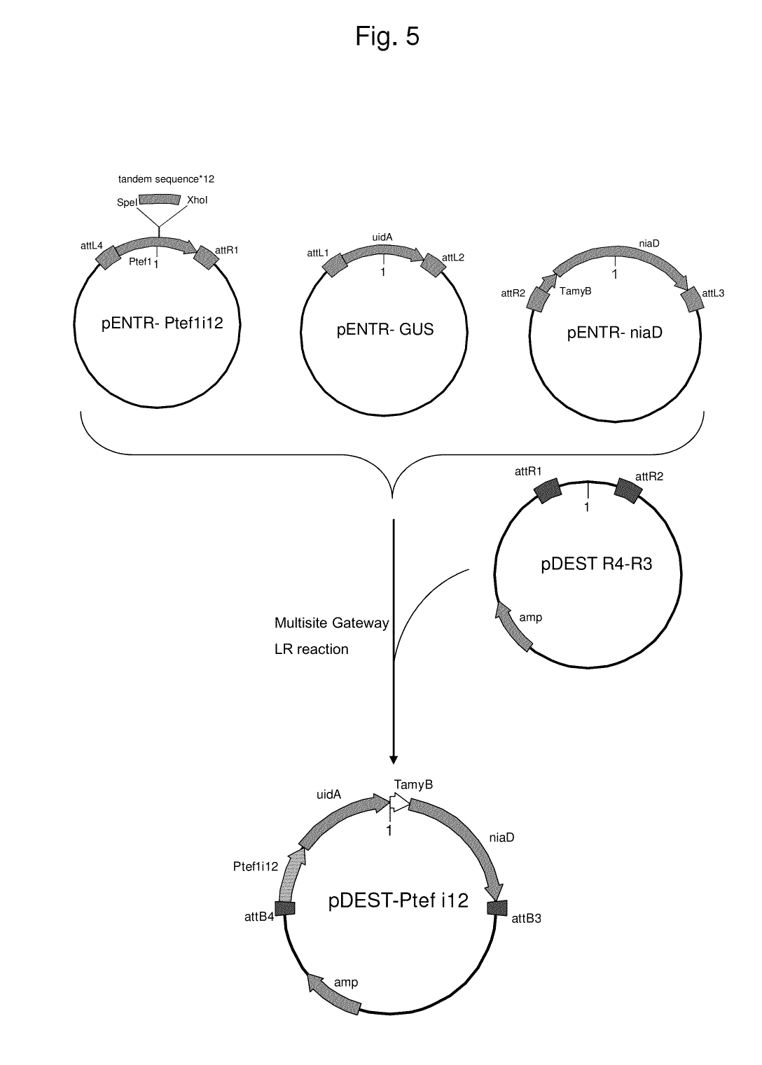

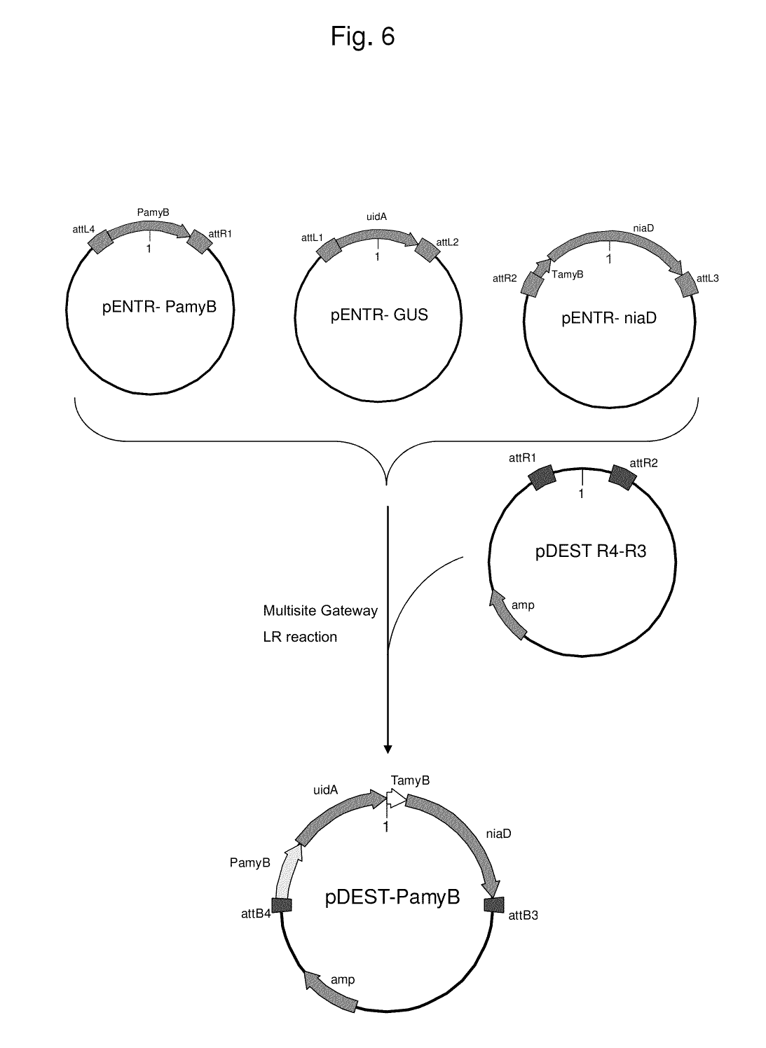

Next, 4 types of gene transfer vector were constructed using MultiSite Gateway (Invitrogen). As the 1^{st} gene transfer vector, as shown in FIG. 3, various entry clones including pENTR-Ptefl, pENTR-GUS, and pENTR-niaD were subjected to LR reaction with pEST R4-R3, so that pDEST-Ptefl was constructed. As the 2^{nd} gene transfer vector, as shown in FIG. 4, various entry clones including pENTR-Ptefli6, pENTR-GUS, and pENTR-niaD were subjected to LR reaction with pEST R4-R3, so that pDEST-Ptefi6 was constructed. As the 3^{rd} gene transfer vector, as shown in FIG. 5, various entry clones including pENTR-Pteflil2, pENTR-GUS, and pENTR-niaD were subjected to LR reaction with pEST R4-R3, so that pDEST-Ptefil2 was constructed. As the 4^{th} gene transfer vector, as shown in FIG. 6, various entry clones including pENTR-PamyB, pENTR-GUS, and pENTR-niaD were subjected to LR reaction with pEST R4-R3, so that pDEST-PamyB was constructed.

(4) Gene Transfer to Koji-Kin Aspergillus oryzae and Selection of Transformants

A. oryzae was transformed by a conventional protoplast-PEG method using the 4 types of gene transfer vector (pDEST-Ptefl, pDEST-Pteflil2, pDEST-Ptefli6, and pDEST-PamyB) constructed in (3) above. In addition, as a host, A. oryzae niaD300 (that is, a nitrate reductase mutant strain (niaD⁻)), was used.

Furthermore, transformants were selected using as an indicator the growth in Czapek-Dox medium (0.2% NaNO₃, 0.1% KH₂PO₄, 0.05% KCl, 0.05% MgSO₄.7H₂O, 2% glucose, pH5.5) containing nitric acid as the sole nitrogen source. Specifically, individual fungi capable of growing in Czapek-Dox medium containing nitric acid as the sole nitrogen source were selected as transformants. From the thus selected multiple transformants, transformants into which 1 copy of the transgene (uidA gene) had been introduced were selected by genomic southern analysis using the uidA gene as a probe. As described above, transformants into which one copy of the pDEST-Ptef, pDEST-Ptefil2, pDEST-Ptefi6, or pDEST-PamyB plasmid had been introduced, were designated as Ptefl, Ptefil2, Ptefi6, and PamyB, respectively.

### (5) Solid Culture and Liquid Culture of Transformants

The transformants Ptefl, Ptefil2, Ptefi6, and PamyB obtained in (4) above were cultured by solid culture and liquid culture as described below, so that enzyme solutions were prepared.

Solid culture was performed by the following method. First, the fluid volume of a seed medium (corn starch (5.6 g), polypeptone (1.8 g), KH₂PO₄ (0.1 g), KCl (0.05 g), MgSO₄.7H₂O (0.15 g), CaCl₂.2H₂O (0.2 g), and distilled water (100 ml)) was adjusted to 20 ml using a 100 ml flask. Conidiospores were inoculated in an appropriate amount and then cultured at 30 degrees C. and 150 rpm for 1 day. Subsequently, 3 ml of the seed medium after culture and 1 ml of 1 M ammonium sulfate solution were added to a 100 ml flask containing 5 g of wheat bran, and then the solution was agitated with a glass bar, followed by 2 days of culture at 30 degrees C. under static conditions. After culture, crude extraction of the enzyme solution was performed under the following conditions. An appropriate amount of mycelia growing on a solid medium was collected using tweezers or the like and then crushed using a mortar while freezing the mycelia with liquid nitrogen. An appropriate amount of 0.1 M phosphate buffer (pH7) was added, and then the resultant was agitated with a vortex or the like. Subsequently, the resultant was subjected to centrifugation in a centrifuge at 15,000 rpm for 1 minute, and then the thus obtained supernatant was used as a stock enzyme solution.

Liquid culture was performed by the following method. First, the fluid volume of a seed medium was adjusted to 20 ml using a 100 ml flask. Conidiospores were inoculated in an appropriate amount and then cultured at 30 degrees C. and 150 rpm for 1 day. Subsequently, 3 ml of the seed medium after culture was inoculated to a bran liquid medium (bran (10 g), ammonium sulfate (0.5 g), KH₂PO₄ (0.5 g), MgSO₄.7H₂O (0.05 g), distilled water 100 ml/500 ml baffled flask), DPY liquid medium (dextrin (2 g), polypeptone (1 g), yeast extract (0.5 g), KH₂PO₄ (0.5 g), MgSO₄.7H₂O (0.05 g), distilled water 100 ml/500 ml baffled flask), or a lactose liquid medium (lactose (10 g), ammonium sulfate (0.5 g), KH₂PO₄ (0.5 g), MgSO₄.7H₂O (0.05 g), distilled water 100 ml/500 ml baffled flask), followed by 3 days of culture at 30 degrees C. and 150 rpm. After culture, crude extraction of the enzyme solution was performed under the following conditions. An appropriate amount of mycelia growing in liquid medium was collected using a Pipetman or the like, and then crushed using a mortar while freezing the resultant with liquid nitrogen. An appropriate amount of 0.1M phosphate buffer (pH7) was added and then the resultant was agitated with a vortex or the like. Subsequently, the resultant was subjected to centrifugation in a centrifuge at 15,000 rpm for 1 minute. The thus obtained supernatant was used as a stock enzyme solution.

### (6) Determination of β Glucuronidase Activity

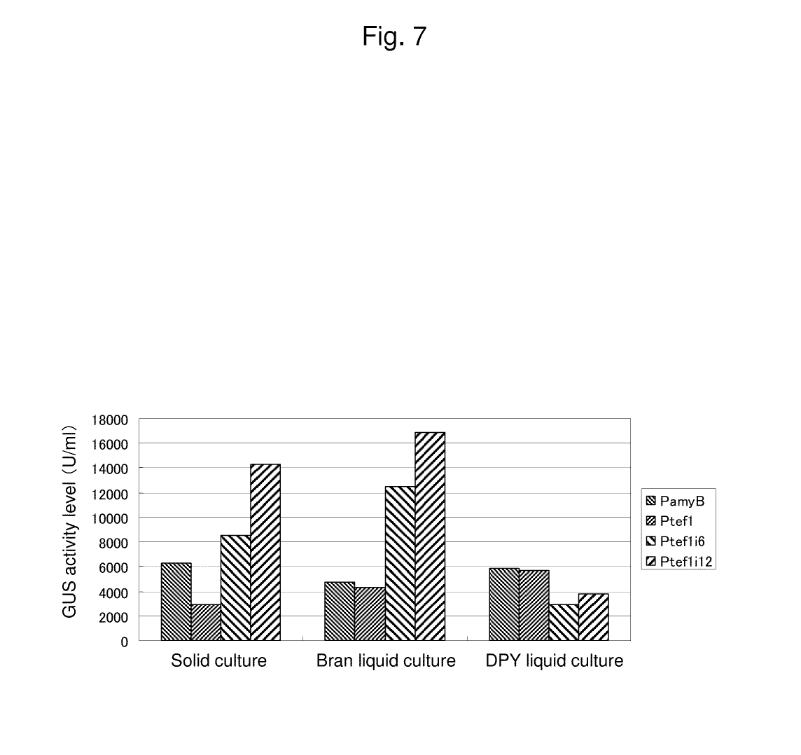

βglucuronidase activity (GUS activity) contained in the stock enzyme solution prepared in (5) above was determined by the method of Jefferson et al. (Proc. Natl. Acad. Sci. U.S.A. 83, 8447-8451). The results are shown in Table 1 and FIG. 7.

TABLE 1

| | GUS activity (U/ml) | | | |
|---|---|---|---|---|
| Transformant | Solid | Bran liquid | DPY liquid | Lactose liquid |
| name | culture | culture | culture | culture |
| PamyB | 6297 | 4712 | 5816 | — |
| Ptef1 | 2908 | 4270 | 5701 | 1996 |
| Ptef1i6 | 8498 | 12523 | 2895 | — |
| Ptef1i12 | 14310 | 16845 | 3829 | 2464 |

As shown in Table 1 and FIG. 7, in solid culture, Pteflil2 exhibited improved GUS activity to a level 4.92 times greater than Ptefl activity and 2.06 times greater than PamyB activity. Furthermore, in solid culture, Ptefli6 exhibited improved GUS activity to a level 2.92 times greater than Ptefl activity and to a level 1.34 times greater than PamyB activity.

Meanwhile, when bran liquid media were used, Pteflil2 exhibited improved GUS activity to a level 3.94 times greater than Ptefl activity and to a level 3.57 times greater than PamyB activity. When bran liquid media were used, Ptefli6 exhibited improved GUS activity to a level 2.93 times greater than Ptefl activity and to a level 2.65 times greater than PamyB activity.

Furthermore, when lactose liquid media were used, Pteflil2 exhibited improved GUS activity to a level 1.23 times greater than Ptefl activity, but the degree of its GUS activity was lower than those exhibited under other culture conditions.

It was confirmed by the above results that under conditions for culture using bran as a substrate, such as solid culture and bran liquid culture conditions, the GUS activity of Pteflil2 was significantly improved compared with that of PamyB, which is generally expressed at a high level. However, in the case of liquid culture using lactose, significant improvement in GUS activity of Pteflil2 was not observed. It was revealed by the results that the cis-acting element designed in the Example is characterized by further improving gene expression with a medium such as a bran medium containing xylan.

Moreover, it was understood based on the results of the Example that when the cis-acting element designed in the Example was repeated 12 times (12 sets), GUS activity was improved to a greater extent than a case in which it was repeated 6 times (6 sets of the cis-acting element).

Meanwhile, Acta. Biochim. Biophys. Sin. (2008): 158-165 clearly describes that a promoter containing an about 200-bp region repeated 4 times therein exhibited improved promoter activity to a level about 1.4 times greater than a promoter having only one 200-bp region, but the activity of a promoter containing the region repeated 6 times therein was almost equivalent to that of a promoter containing the region repeated 4 times therein. As described above, the about 200-bp region disclosed in Acta. Biochim. Biophys. Sin. (2008): 158-165 was confirmed to have an effect of improving gene expression, but the degree was evaluated to be low. Furthermore, although the effect of improving gene expression is further increased with the use of the about 200-bp region repeated 4 times as disclosed in Acta. Biochim. Biophys. Sin. (2008): 158-165, the effect of improving gene expression is not further increased if the region is repeated more than 4 times.

As described above, the cis-acting element designed in the Example exhibits an effect of improving gene expression that is significantly greater than that of the conventionally known cis-acting element (the about 200-bp region disclosed in Acta. Biochim. Biophys. Sin. (2008): 158-165). Furthermore, unlike the conventionally known cis-acting element (the about 200-bp region disclosed in Acta. Biochim. Biophys. Sin. (2008): 158-165), the cis-acting element designed in this Example can enhance the effect of improving gene expression depending on the number of repetitions, even if it is used in tandem in a greater number of sets thereof. Therefore, in the case of the cis-acting element designed in this Example, the effect of improving gene expression can be regulated more precisely by appropriately setting the number of repetitions.

All publications, patents, and patent applications cited herein are incorporated herein by reference in their entirety.

1.-18. (canceled)

19. A method for producing a substance, comprising:
culturing a transformant comprising a cis-acting element that has a region in which the XlnR/Ace2-binding sequence (ggctaa) and the Hap complex-binding sequence (ccaat) are arranged with a spacer sequence of 0 to 100 nucleotides between them, wherein the cis-acting element is incorporated into a site upstream of a promoter region in a desired gene, in at least one medium selected from a medium containing an herbaceous species as a raw material, a medium containing woody species as a raw material, and a medium containing an agricultural product residue or waste as a raw material; and
recovering a target substance from the medium and/or the transformant after culture.

20. The method for producing a substance according to claim 19, wherein the transformant has a cis-acting element wherein the region in which the XlnR/Ace2-binding sequence (ggctaa) and the Hap complex-binding sequence (ccaat) are arranged with a spacer sequence of 0 to 100 nucleotides between them, is repeated multiple times via linker sequences.

21. The method for producing a substance according to claim 20, wherein the number of repetitions of the region ranges from 1 to 50.

22. The method for producing a substance according to claim 19, wherein the transformant comprises the desired gene containing a foreign gene.

23. The method for producing a substance according to claim 19, wherein a host cell of the transformant is a filamentous fungus.

24. The method for producing a substance according to claim 19, wherein the target substance is a protein encoded by a gene, in which the expression of the gene is enhanced by the cis-acting element.


SEQUENCE LISTING (12 sequences)

SEQ ID NO: 1 - DNA, 111 bp, Artificial Sequence
  synthetic DNA
ggctaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccaat

SEQ ID NO: 2 - DNA, 111 bp, Artificial Sequence
  synthetic DNA
ccaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggctaa

SEQ ID NO: 3 - DNA, 26 bp, Artificial Sequence
  Synthetic DNA
nnnggctaannnnnnccaatnnnnnn

SEQ ID NO: 4 - DNA, 26 bp, Artificial Sequence
  Synthetic DNA
ttaggctaaacgtacccaatgataag

SEQ ID NO: 5 - DNA, 48 bp, Artificial Sequence
  Synthetic DNA
ggggacaactttgtatagaaaagttgtttctagatagcgagagtaaaa

SEQ ID NO: 6 - DNA, 49 bp, Artificial Sequence
  Synthetic DNA
ggggactgcttttttgtacaaacttggtttgaaggtggtgcgaactttg

SEQ ID NO: 7 - DNA, 50 bp, Artificial Sequence
  Synthetic DNA
ggggacaactttgtatagaaaagttgttccagtgaattcatggtgttttg

SEQ ID NO: 8 - DNA, 50 bp, Artificial Sequence
  Synthetic DNA
ggggactgcttttttgtacaaacttggaaatgccttctgtggggtttatt

SEQ ID NO: 9 - DNA, 21 bp, Artificial Sequence
  Synthetic DNA
atgttacgtcctgtagaaacc

SEQ ID NO: 10 - DNA, 21 bp, Artificial Sequence
  Synthetic DNA
tcattgtttgcctccctgctg

SEQ ID NO: 11 - DNA, 49 bp, Artificial Sequence
  Synthetic DNA
ggggacagctttcttgtacaaagtgggtgatctgtagtagctcgtgaag

SEQ ID NO: 12 - DNA, 49 bp, Artificial Sequence
  Synthetic DNA
ggggacaactttgtataataaagttggaagctttggatttcctacgtct